(19) United States
(12) Patent Application Publication
(10) Pub. No.: US 2022/0378495 A1
(43) Pub. Date: Dec. 1, 2022

(54) ELECTROSURGICAL FORCEPS WITH TENSION SENSOR

(71) Applicant: Covidien LP, Mansfield, MA (US)

(72) Inventors: James R. Fagan, Erie, CO (US); Barbara R. Tyrrell, Erie, CO (US)

(21) Appl. No.: 17/691,286

(22) Filed: Mar. 10, 2022

Related U.S. Application Data
(60) Provisional application No. 63/193,789, filed on May 27, 2021.

(51) Int. Cl. A61B 18/14; A61B 90/00

(52) U.S. Cl. CPC A61B 18/1445; A61B 90/06; A61B 2018/00178

(57) ABSTRACT

A surgical instrument includes a housing having an elongated shaft extending distally from the housing and configured to support an end effector assembly at a distal end thereof. The end effector assembly including first and second jaw members, each jaw member including a tissue sealing plate disposed thereon. A force gauge is disposed on one or both the tissue sealing plates of the first and second jaw members. The force gauge is configured to measure a force associated with the one (or both) tissue sealing plate and communicate the force measurement to an electrosurgical energy source. The electrosurgical energy source is configured to correlate the force measurement to an amount of tension on tissue disposed between the first and second jaw members and alert a user if the amount of tension falls outside a particular range.

### BACKGROUND

### 1. Technical Field

The present disclosure relates generally to the field of surgical instruments. In particular, the disclosure relates to electrosurgical forceps that includes a tension sensor to measure the amount of tension on tissue prior to or during activation.

### 2. Background of Related Art

Instruments such as electrosurgical forceps are commonly used in open and endoscopic surgical procedures to coagulate, cauterize and seal tissue. Such forceps typically include a pair of jaw members that can be controlled by a surgeon to grasp targeted tissue, such as, e.g., a blood vessel. The jaw members may be approximated to apply a mechanical clamping force to the tissue, and are associated with at least one electrode to permit the delivery of electrosurgical energy to the tissue. The combination of the mechanical clamping force and the electrosurgical energy has been demonstrated to join adjacent layers of tissue captured between the jaw members. When the adjacent layers of tissue include the walls of a blood vessel, sealing the tissue may result in hemostasis, which may facilitate the transection of the sealed tissue. A detailed discussion of the use of an electrosurgical forceps may be found in U.S. Pat. No. 7,255,697 to Dycus et al.

A bipolar electrosurgical forceps typically includes opposed electrodes disposed on clamping faces of the jaw members. The electrodes are charged to opposite electrical potentials such that an electrosurgical current may be selectively transferred through tissue grasped between the electrodes. To effect a proper seal, particularly in relatively large vessels, two predominant mechanical parameters must be accurately controlled; the pressure applied to the vessel, and the gap distance established between the electrodes.

Both the pressure and gap distance influence the effectiveness of the resultant tissue seal. If an adequate gap distance is not maintained, there is a possibility that the opposed electrodes will contact one another, which may cause a short circuit and prevent energy from being transferred through the tissue. Also, if too low a force is applied the tissue may have a tendency to move before an adequate seal can be generated. The thickness of a typical effective tissue seal is optimally between about 0.001 and about 0.006 inches. Below this range, the seal may shred or tear and above this range the vessel walls may not be effectively joined. Closure pressures for sealing large tissue structures preferably fall within the range of about 3 kg/cm² to about 16 kg/cm².

Many endoscopic surgical instruments utilize handle or levers to actuate the end effector assembly typically disposed at a distal end of the instrument. For example, actuation of the handle correspondingly actuates the jaw members. Once closed about tissue electrical energy is delivered to treat tissue. With open forceps, two opposing handles are pivotable relative to tone another to grasp tissue prior to energizing the jaw members.

In some instances, the surgeon may manipulate the tissue prior to activation to provide tension on the tissue to facilitate a tissue seal or separation after a tissue seal. For example, the surgeon may slightly rotate the housing or opposing handles when grasping tissue to induce a torque on the tissue or vessel, or the surgeon may pull/push the tissue in a certain direction to place tension on the tissue prior to activation.

### SUMMARY

As used herein, the term “distal” refers to the portion of the instrument or component thereof that is being described that is further from a user, while the term “proximal” refers to the portion of the instrument or component thereof that is being described that is closer to a user. Further, to the extent consistent, any of the aspects described herein may be used in conjunction with any of the other aspects described herein. As used herein the term “tissue” is meant to include variously-sized vessels.

Provided in accordance with aspects of the present disclosure is a surgical instrument which includes a housing having an elongated shaft extending distally from the housing and configured to support an end effector assembly at a distal end thereof. The end effector assembly including first and second jaw members, each jaw member including a tissue sealing plate disposed thereon. A force gauge is disposed on one or both of the tissue sealing plates of the first and second jaw members. The force gauge is configured to measure a force associated with the one (or both) tissue sealing plate and communicate the force measurement to an electrosurgical energy source. The electrosurgical energy source is configured to correlate the force measurement to an amount of tension on tissue disposed between the first and second jaw members and alert a user if the amount of tension falls outside a particular range.

In aspects according to the present disclosure, an indicator is disposed on the end effector and provides feedback to the user regarding the amount of tension on the tissue. In other aspects according to the present disclosure, the indicator is at least one of audible or visual.

In aspects according to the present disclosure, the force gauge is disposed on a non-tissue engaging side of the at least one tissue sealing plate and extends therealong.

In aspects according to the present disclosure, the force gauge is configured to measure a longitudinal force relative to a longitudinal axis extending along the at least one tissue sealing plate and communicate the measurement of the longitudinal force to the electrosurgical energy source. In other aspects according to the present disclosure, the force gauge is configured to measure a rotational force relative a longitudinal axis extending along the at least one tissue sealing plate and communicate the measurement of the rotational force to the electrosurgical energy source.

Provided in accordance with aspects of the present disclosure is a surgical instrument which includes a housing having an elongated shaft extending distally from the housing and configured to support an end effector assembly at a distal end thereof. The end effector assembly including first and second jaw members, each jaw member including a tissue sealing plate disposed thereon. A spring-like force gauge is configured to support one or both tissue sealing plates thereon. The spring-like force gauge includes a plate having a series of springs disposed therein configured to measure a force associated with the one (or both) tissue sealing plate and communicate the force measurement to an electrosurgical energy source. The electrosurgical energy source is configured to correlate the force measurement to an amount of tension on tissue disposed between the first and second jaw members and alert a user if the amount of tension falls outside a particular range.

In aspects according to the present disclosure, an indicator is disposed on the end effector and provides feedback to the user regarding the amount of tension on the tissue. In other aspects according to the present disclosure, the indicator is at least one of audible or visual.

In aspects according to the present disclosure, the spring-like force gauge supports the entire at least one sealing plate.

In aspects according to the present disclosure, the spring-like force gauge is configured to measure a longitudinal force relative to a longitudinal axis extending along the at least one tissue sealing plate and communicate the measurement of the longitudinal force to the electrosurgical energy source.

In aspects according to the present disclosure, the force gauge is configured to measure a rotational force relative to a longitudinal axis extending along the at least one tissue sealing plate and communicate the measurement of the rotational force to the electrosurgical energy source.

### BRIEF DESCRIPTION OF THE DRAWINGS

The accompanying drawings, which are incorporated in and constitute a part of this specification, illustrate embodiments of the present disclosure and, together with the detailed description of the embodiments given below, serve to explain the principles of the disclosure.

FIGS. 1A-1C are views of an endoscopic electrosurgical forceps including a housing, an elongated shaft, an end effector and one embodiment of a force gauge extending along the end effector;

FIGS. 2A-2B are views of an open electrosurgical forceps including opposing shafts and an end effector assembly at a distal end thereof;

FIG. 3A is an enlarged, perspective distal end view of the end effector of the endoscopic electrosurgical forceps of FIG. 1A including a force gauge in accordance with another embodiment of the present disclosure;

FIG. 3B is a view taken along line 3B-3B of FIG. 3A including a schematic illustration of the connection to an electrosurgical energy source; and

FIG. 3C is a view similar to FIG. 3B showing rotational force being applied to the end effector.

### DETAILED DESCRIPTION

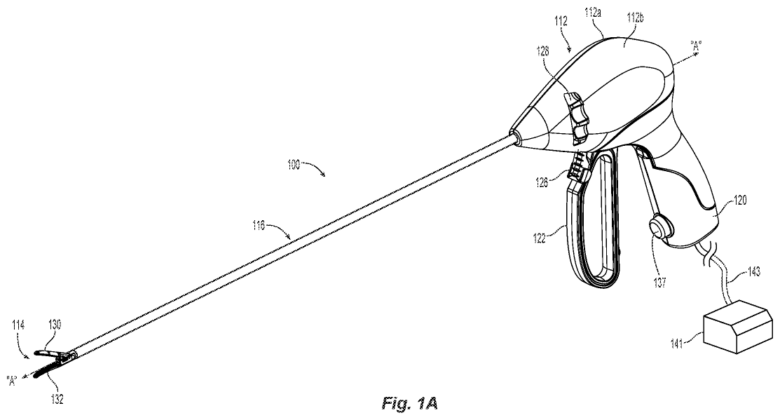

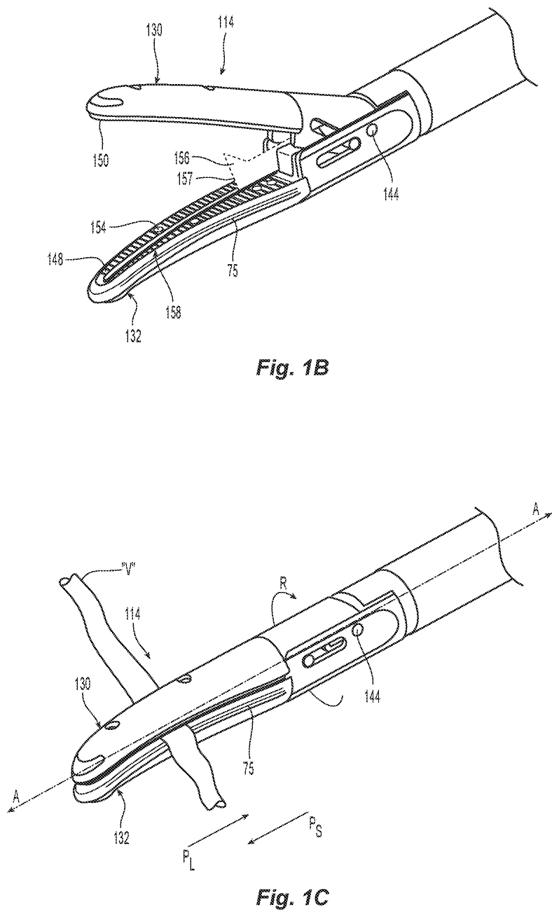

Referring initially to FIG. 1A, an endoscopic electrosurgical forceps 100 generally includes a housing 112 that supports various actuators thereon for remotely controlling an end effector 114 through an elongated shaft 116. Although this configuration is typically associated with instruments for use in laparoscopic or endoscopic surgical procedures, various aspects of the present disclosure may be practiced with traditional open instruments and in connection with endoluminal procedures as well (See FIG. 2A). The housing 112 is constructed of a left housing half 112a and a right housing half 112b. The left and right designation of the housing halves 112a, 112b refer to the respective directions as perceived by an operator using the forceps 100. The housing halves 112a, 112b may be constructed of sturdy plastic, and may be joined to one another by adhesives, ultrasonic welding or other suitable assembly methods.

To mechanically control the end effector 114, the housing 112 supports a stationary handle 120, a movable handle 122, a trigger 126 and a rotation knob 128. The movable handle 122 is operable to move the end effector 114 between an open configuration wherein a pair of opposed jaw members 130, 132 are disposed in spaced relation relative to one another, and a closed or clamping configuration wherein the jaw members 130, 132 are closer together. Approximation of the movable handle 122 with the stationary handle 120 serves to move the end effector 114 to the closed configuration and separation of the movable handle 122 from the stationary handle 120 serves to move the end effector 114 to the open configuration. The trigger 126 is operable to extend and retract a knife blade 156 (FIG. 1B) through the end effector 114 when the end effector 114 is in the closed configuration. The rotation knob 128 serves to rotate the elongated shaft 116 and the end effector 114 about a longitudinal axis A-A extending through the forceps 100.

To electrically control the end effector 114, the stationary handle 120 supports a depressible button 137 thereon, which is operable by the user via movable handle 122 to initiate and terminate the delivery of electrosurgical energy to the end effector 114. The depressible button 137 is mechanically coupled to a switch (not shown) disposed within the stationary handle 120 which is in electrical communication with an electrosurgical generator 141 via suitable electrical wiring (not explicitly referenced) extending from the housing 112 through a cable 143 extending between the housing 112 and the electrosurgical generator 141. The generator 141 may include devices such as the LigaSure® Vessel Sealing Generator and the ForceTriad® Generator sold by Medtronic. The cable 143 may include a connector (not shown) thereon such that the forceps 100 may be selectively coupled electrically to the generator 141.

As mentioned above, the end effector 114 may be moved from the open configuration (FIG. 1B) wherein tissue (not shown) is received between the jaw members 130, 132, and the closed configuration (FIG. 1C), wherein the tissue is clamped and treated. The jaw members 130, 132 pivot about a pivot pin 144 (FIG. 1B) to move the end effector 114 to the closed configuration (FIG. 1C) of wherein sealing plates 148, 150 associated with respective jaw members 132, 130 provide a pressure to tissue grasped therebetween. In some embodiments, to provide an effective tissue seal, a pressure within a range between about 3 kg/cm² to about 16 kg/cm² and, desirably, within a working range of about 7 kg/cm² to about 13 kg/cm², may be applied to the tissue. Also, in the closed configuration, a separation or gap distance is maintained between the sealing plates 148, 150 by an array of stop members 154 (FIG. 1B) disposed on or adjacent the sealing plates 148, 150. The stop members 154 contact opposing surfaces on the opposing jaw member 130, 132 and prohibit further approximation of the sealing plates 148, 150. In some embodiments, to provide an effective tissue seal, an appropriate gap distance of about 0.001 inches to about 0.010 inches and, desirably, between about 0.002 inches to about 0.005 inches, may be provided. In some embodiments, the stop members 154 are constructed of a heat-resistant ceramic deposited onto the jaw members 130, 132. In other embodiments, the stop members 154 are constructed of an electrically non-conductive plastic molded onto the jaw members 130, 132, e.g., by a process such as overmolding or injection molding.

The upper and lower jaw members 130, 132 are electrically coupled to cable 143, and thus to the generator 141 (e.g., via respective suitable electrical wiring extending through the elongated shaft 116) to provide an electrical pathway to a pair of electrically conductive, tissue-engaging sealing plates 148, 150 disposed on the lower and upper jaw members 132, 130, respectively. The sealing plate 148 of the lower jaw member 132 opposes the sealing plate 150 of the upper jaw member 130. In some embodiments, the sealing plates 148 and 150 are electrically coupled to opposite terminals, e.g., positive or active (+) and negative or return (—) terminals associated with the generator 141. Thus, bipolar energy may be provided through the sealing plates 148 and 150 to tissue.

Alternatively, the sealing plates 148 and 150 may be configured to deliver monopolar energy to tissue. In a monopolar configuration, one or both sealing plates 148 and 150 deliver electrosurgical energy from an active terminal, e.g., (+), while a return pad (not shown) is placed generally on a patient and provides a return path to the opposite terminal, e.g., (−), of the generator 141. Each jaw member 130, 132 includes a jaw insert (not shown) and an insulator (not shown) that serves to electrically insulate the sealing plates 150, 148 from the jaw insert of the jaw members 130, 132, respectively.

Electrosurgical energy may be delivered to the tissue through the electrically conductive seal plates 148, 150 to effect a tissue seal. Once a tissue seal is established, the knife blade 156 having a sharpened distal edge 157 may be advanced through a knife channel 158 defined in one or both jaw members 130, 132 and respective seal plates 150, 148 to transect the sealed tissue. Although the knife blade 156 is depicted in FIG. 1B as extending from the elongated shaft 116 when the end effector 114 is in an open configuration, in some embodiments, extension of the knife blade 156 into the knife channel 158 when the end effector 114 is in the open configuration may be prevented by one or more lockout features. An electrosurgical knife (not shown) may also be utilized to cut tissue. For example, U.S. Provisional Patent Application Ser. No. 63/056,113 filed Jul. 24, 2020 describes one such electrosurgical cutter and is incorporated by reference in its entirety herein.

Referring now to FIGS. 2A-2B, an open forceps 10 contemplated for use in connection with traditional open surgical procedures is shown. For the purposes herein, either an open instrument, e.g., forceps 10, or an endoscopic instrument (FIGS. 1A-1C) may be utilized in accordance with the present disclosure. Obviously, different electrical and mechanical connections and considerations apply to each particular type of instrument; however, the novel aspects with respect to the end effector assembly and its operating characteristics remain generally consistent with respect to both the open and endoscopic configurations.

With continued reference to FIGS. 2A-2B, forceps 10 includes two elongated shafts 12a and 12b, each having a proximal end 14a and 14b, and a distal end 16a and 16b, respectively. Forceps 10 further includes an end effector assembly 200 attached to distal ends 16a and 16b of shafts 12a and 12b, respectively. End effector assembly 200 includes a pair of opposing jaw members 210, 220 that are pivotably connected about a pivot 203. Each shaft 12a and 12b includes a handle 17a and 17b disposed at the proximal end 14a and 14b thereof. Each handle 17a and 17b defines a finger hole 18a and 18b therethrough for receiving a finger of the user. Finger holes 18a and 18b facilitate movement of the shaft members 12a and 12b relative to one another between a spaced-apart position and an approximated position, which, in turn, pivot jaw members 210, 220 from an open position, wherein the jaw members 210, 220 are disposed in spaced-apart relation relative to one another, to a closed position, wherein the jaw members 210, 220 cooperate to grasp tissue therebetween.

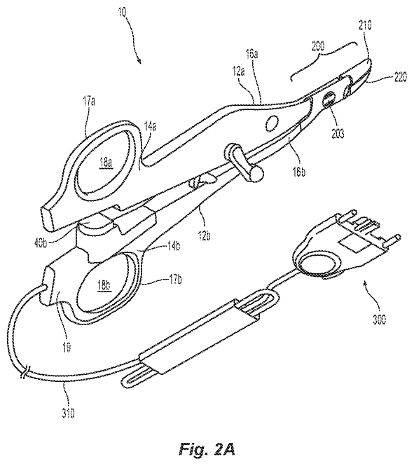

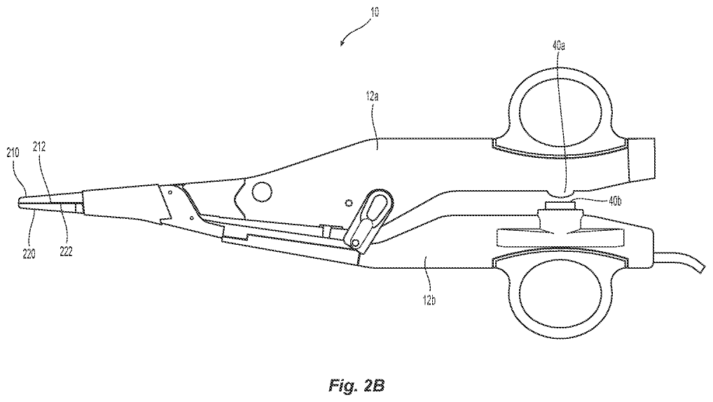

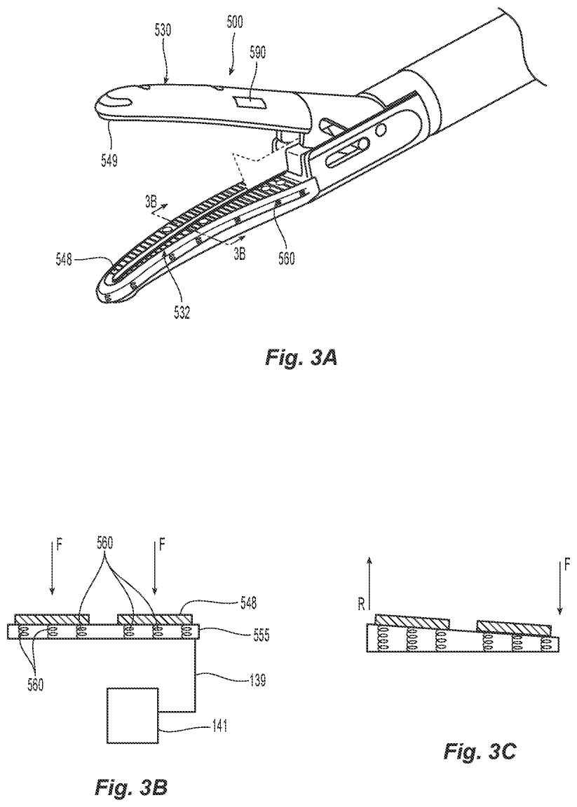

Continuing with reference to FIGS. 2A-2B, one of the shafts, e.g., shaft 12b, includes a proximal shaft connector 19 that is designed to connect the forceps 10 to a source of electrosurgical energy such as an electrosurgical generator 141 (FIGS. 1A, 3B). Proximal shaft connector 19 secures an electrosurgical cable 310 to forceps 10 such that the user may selectively apply electrosurgical energy to electrically-conductive plates 212, 222 (See FIG. 2B) of jaw members 210, 220, respectively.

More specifically, cable 310 includes a plurality of wires (not shown) extending therethrough that has sufficient length to extend through one of the shaft members, e.g., shaft member 12b, in order to provide electrical energy to the conductive plates 212, 222 of jaw members 210, 220, respectively, of end effector assembly 200, e.g., upon activation of activation switch 40b (See FIGS. 2A and 2B). Other types activation switches are also contemplated, e.g., finger switch, toggle switch, foot switch, etc. and may be configured for this purpose. Cable 310 operably connects to generator 141 via plug 300.

Activation switch 40b is disposed at proximal end 14b of shaft member 12b and extends therefrom towards shaft member 12a. A corresponding surface 40a (FIG. 2B) is defined along shaft member 12a toward proximal end 14a thereof and is configured to actuate activation switch 40b. More specifically, upon approximation of shaft members 12a, 12b, e.g., when jaw members 210, 220 are moved to the closed position, activation switch 40b is moved into contact with, or in close proximity of surface 40a. Upon further approximation of shaft members 12a, 12b, e.g., upon application of a pre-determined closure force to jaw members 210, 220, activation switch 40b is advanced further into surface 40a to depress activation switch 40b. Activation switch 40b controls the supply of electrosurgical energy to jaw members 210, 220 such that, upon depression of activation switch 40b, electrosurgical energy is supplied to conductive surface 212 and/or conductive surface 222 of jaw members 210, 220, respectively, to seal tissue grasped therebetween. The electrical energy may be energy supplied through a proprietary Ligasure® sealing algorithm owned by Medtronic. The switch 40b may be disposed on either shaft 12a, 12b.

Referring back to FIG. 1C, once a vessel “V” or tissue is clamped between jaw members 130, 132 of end effector 114, the surgeon is ready to seal and cut the vessel “V” upon activation of the energy activation switch, e.g., switch 137, of endoscopic forceps 100. The switch 40b of open forceps 10 may be used for similar purposes, however, for the purposes herein, forceps 100 will be described hereinbelow.

One (or both) of the jaw members 130, 132 includes an elongated force gauge 75 disposed along one or both sides thereof configured to provide feedback relating to the force being applied on the seal plate 148 and/or seal plate 150. Force gauge 75 may be sized according the size of the sealing surface, e.g., sealing plate 148, and extend along a substantial portion thereof.

Alternatively, force gauge 75 may be configured as a cumulation of multiple force gauges that, cooperatively, provide force feedback relating the to seal plate 148 and/or seal plate 150. Moreover, the force gauge 75 may be arranged in a matrix-like fashion such that the force gauge 75 can provide feedback in more than one direction, e.g., along the seal plate in the direction of the longitudinal axis A-A, transverse to the longitudinal axis A-A, rotationally about the longitudinal axis A-A, and/or normal to the longitudinal axis A-A.

More particularly, FIG. 1C shows the force gauge 75 disposed on the outer peripheral edge of the seal plate 148 and extending therealong. The force gauge 75 is configured to connect to an electrical source the generator 141 regarding the applied force on the seal plate 148 which is directly attributable or may be correlated to the applied tension on the vessel “V” or tissue. For example, if the end effector 114 (or shaft 116) is rotated in the direction “R” when a vessel “V” is grasped between the jaw members 130, 132 to provide additional tension to facilitate sealing and/or cutting, the force gauge 75 may detect a rotational force on the seal plate 148 which may be attributed to a corresponding tension of the vessel “V” disposed between the jaw members 130, 132. For example, the reading from the force gauge 75 is relayed to the generator 141 and a corresponding tension is determined via a look up table or mathematical equation.

The amount of tension on the vessel “V” may fall outside of an acceptable parameter and, as a result, energy delivery to the end effector 114 may be prevented prior to activation, may be terminated if already active, or an alarm may sound to alert the user that too much tension is being applied to the vessel “V”. The tension feedback may be as sophisticated as a continuous tension reading on the generator 141 or as simple as a high tension alarm.

In addition to detecting rotational forces on the seal plate 148 which correlates to a corresponding tension on the vessel “V”, the same or a different force gauge 75 may be positioned to detect multiple movements of the end effector 114 which may cause too much tension on the vessel “V”, e.g., pushing or pulling the vessel “V”. For example, as shown in FIG. 1C, distal or pushing movement along the longitudinal axis A-A in the forward, pushing direction “PS” may result in additional tension being placed on the vessel “V” along with pulling or proximal movement in direction “PL”. The force gauge 75 may be configured to specify the direction of the tension on the vessel “V” providing the surgeon with a visual representation of the associated forces and allowing for correction if necessary or to satisfy an alarm condition.

In embodiments, the force gauge 75 may be disposed on other areas of the end effector 114 to measure the tension on the vessel “V” during manipulation of the forceps 100.

FIGS. 3A-3B show another embodiment of an end effector 500 according to the present disclosure having a spring-like force gauge 575 for use with detecting vessel “V” tension. Tissue sealing plate 548 is supported atop a spring-like plate 555 having a series of springs 560 disposed therein. Alternatively, spring-like plate 555 may be composed of a compressible material having similar characteristics to springs 560. The springs 560 are arranged to support the sealing plate 548 in a balanced fashion relative to the opposing sealing plate 549 on jaw member 530 along and across the same.

When a vessel “V” is grasped between the jaw members 530, 532 and compressed under the appropriate forces for sealing vessels “V”, the spring-like plate 555 compresses against the force of the spring 560 in a generally even fashion. When a surgeon attempts to tension the vessel “V” by manipulating the vessel “V” in a given direction (e.g., “tenting), the spring-like 555 plate will compress under the manipulation force sending a signal back to the generator 141 for conversion into a corresponding tension on the vessel “V” (as mentioned above). When the manipulation force and resultant tension on the vessel “V” fall outside of a specified value, energy delivery to the end effector 500 may be prevented prior to activation, may be terminated if already active, or an alarm may sound to alert the user that too much tension is being applied to the vessel “V”.

For example, with respect to FIG. 3B, the surgeon may opt to pull the vessel “V” upwardly (e.g., away from the body) prior to activation to tension the tissue for sealing and cutting. Pulling the vessel “V” upwardly will place an increased force “F” on the tissue sealing plate 548 and spring-like plate 555 thereby compressing the springs 560 and sending a signal to the generator 141 via lead 139. The generator 141 evaluates the force signal and associates a corresponding tension to the vessel “V”. If outside a preferred range, the generator 141 alerts the user, does not allow activation or terminates energy depending upon the state of the forceps 100. As mentioned above, various visual or audible indicators or displays may be employed depending upon a particular purpose. A visual or audible indicator 590 may be associated with the end effector (See end effector 500, FIG. 3A) or the generator 141.

Similarly, with respect to FIG. 3C, the surgeon may opt to rotate the vessel “V” in the direction “R” prior to activation to tension the tissue for sealing and cutting. Rotating the vessel “V” will place an increased force “F” on the tissue sealing plate 548 and spring-like plate 555 thereby compressing the springs 560 and sending a signal to the generator 141 via lead 139. Similar to above, the generator 141 evaluates the force signal and associates a corresponding tension to the vessel “V”. If outside a preferred range, the generator 141 alerts the user, does not allow activation or terminates energy depending upon the state of the forceps 100. The visual or audible indicator 590 associated with the end effector 500 or the generator 141 provides feedback to the user during manipulation of the forceps 100.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as examples of particular embodiments. Those skilled in the art will envision other modifications within the scope and spirit of the claims appended hereto.

Although the foregoing disclosure has been described in some detail by way of illustration and example, for purposes of clarity or understanding, it will be obvious that certain changes and modifications may be practiced within the scope of the appended claims.

What is claimed is:

1. A surgical instrument, comprising:
a housing;
an elongated shaft extending distally from the housing and configured to support an end effector assembly at a distal end thereof, the end effector assembly including first and second jaw members, each jaw member including a tissue sealing plate disposed thereon; and
a force gauge disposed on at least one of the tissue sealing plates of the first and second jaw members, the force gauge configured to measure a force associated with the at least one tissue sealing plate and communicate the force measurement to an electrosurgical energy source, the electrosurgical energy source configured to correlate the force measurement to an amount of tension on tissue disposed between the first and second jaw members and alert a user if the amount of tension falls outside a particular range.

2. The surgical instrument according to claim 1, wherein an indicator is disposed on the end effector and provides feedback to the user regarding the amount of tension on the tissue.

3. The surgical instrument according to claim 2, wherein the indicator is at least one of audible or visual.

4. The surgical instrument according to claim 1, wherein the force gauge is disposed on a non-tissue engaging side of the at least one tissue sealing plate and extends therealong.

5. The surgical instrument according to claim 1, wherein the force gauge is configured to measure a longitudinal force relative to a longitudinal axis extending along the at least one tissue sealing plate and communicate the measurement of the longitudinal force to the electrosurgical energy source.

6. The surgical instrument according to claim 1, wherein the force gauge is configured to measure a rotational force relative a longitudinal axis extending along the at least one tissue sealing plate and communicate the measurement of the rotational force to the electrosurgical energy source.

7. A surgical instrument, comprising:
a housing;
an elongated shaft extending distally from the housing and configured to support an end effector assembly at a distal end thereof, the end effector assembly including first and second jaw members, each jaw member including a tissue sealing plate disposed thereon; and
a spring-like force gauge configured to support at least a portion of at least one of the tissue sealing plates thereon, the spring-like force gauge including a plate having a series of springs disposed therein configured to measure a force associated with the at least one tissue sealing plate and communicate the force measurement to an electrosurgical energy source, the electrosurgical energy source configured to correlate the force measurement to an amount of tension on tissue disposed between the first and second jaw members and alert a user if the amount of tension falls outside a particular range.

8. The surgical instrument according to claim 7, wherein an indicator is disposed on the end effector and provides feedback to the user regarding the amount of tension on the tissue.

9. The surgical instrument according to claim 8, wherein the indicator is at least one of audible or visual.

10. The surgical instrument according to claim 7, wherein the spring-like force gauge supports the entire at least one sealing plate.

11. The surgical instrument according to claim 7, wherein the spring-like force gauge is configured to measure a longitudinal force relative to a longitudinal axis extending along the at least one tissue sealing plate and communicate the measurement of the longitudinal force to the electrosurgical energy source.

12. The surgical instrument according to claim 7, wherein the force gauge is configured to measure a rotational force relative to a longitudinal axis extending along the at least one tissue sealing plate and communicate the measurement of the rotational force to the electrosurgical energy source.
